(19) United States
(12) Patent Application Publication
(10) Pub. No.: US 2016/0075619 A1
(43) Pub. Date: Mar. 17, 2016

(54) Process for Recovering Olefins from Manufacturing Operations

(71) Applicant: Paul Su, Saratoga, CA (US)

(72) Inventor: Paul Su, Saratoga, CA (US)

(73) Assignee: MEMBRANE TECHNOLOGY AND RESEARCH, INC., Newark, CA (US)

(21) Appl. No.: 14/938,546

(22) Filed: Nov. 11, 2015

Related U.S. Application Data
(63) Continuation-in-part of application No. 14/722,738, filed on May 27, 2015, now Pat. No. 9,216,931.
Application No. 14/722,738 is a continuation-in-part of application No. 14/486,382, filed on Sep. 15, 2014, now Pat. No. 9,073,808.
Application No. 14/486,382 is a continuation-in-part of application No. 14/789,166, filed on Jul. 1, 2015.
Application No. 14/789,166 is a continuation-in-part of application No. 14/486,382, filed on Sep. 15, 2014, now Pat. No. 9,073,808.

(51) Int. Cl. C07C 7/00; C07C 7/144; B01D 71/02; C07C 7/09; B01D 53/00; B01D 53/22

(52) U.S. Cl. CPC C07C 7/005; B01D 53/002; B01D 53/229; B01D 53/226; B01D 71/02; C07C 7/09; C07C 7/144

(57) ABSTRACT

A process for treating an effluent gas stream arising from a manufacturing operation that produces an olefin or an olefin derivative. The process involves compressing the feed gas stream, which comprises an olefin, a paraffin, and a third gas, to produce a compressed stream, then cooling and condensing the compressed stream. The condensation step produces a liquid condensate and an uncondensed gas stream. The liquid condensate is then passed through a membrane separation step. The membrane separation of the condensate results in an olefin-enriched stream, which may be recycled for use within the manufacturing operation, and an olefin-depleted stream, which may be purged from the process.

### CROSS-REFERENCE TO RELATED APPLICATIONS

This application is a continuation-in-part of U.S. application Ser. No. 14/722,738, filed on May 27, 2015, and is a continuation-in-part of U.S. application Ser. No. 14/789,166, filed on Jul. 1, 2015, which are both continuation-in-parts of U.S. application Ser. No. 14/486,382, filed Sep. 15, 2014, which issued as U.S. Pat. No. 9,073,808 on Jul. 7, 2015, the disclosures of all of which are hereby incorporated herein by reference in their entireties.

### FIELD OF THE INVENTION

The invention relates to a process for recovering olefins from a manufacturing operation. More specifically, the invention relates to treating an effluent gas stream using membranes for recovering olefin and separating paraffin.

### BACKGROUND OF THE INVENTION

Olefins, such as ethylene and propylene, and their non-polymeric derivatives, such as isopropyl alcohol and cumene, account for some of the most demanded chemicals in the world. For example, the United States alone produces more than 10 billion pounds of chemicals derived from propylene annually.

Olefins are commonly produced by cracking hydrocarbon feedstocks or catalytically converting oxygenate feedstocks. Traditional methods for cracking include steam cracking, whereby naphtha or other hydrocarbons are reacted with steam to make light olefins, and fluid catalytic cracking (FCC), which is the refinery operation that breaks down larger hydrocarbons to produce naphtha-light components for gasoline, as well as olefins and heating oils. The conventional conversion of oxygenate feedstocks includes methanol-to-olefin (MTO) and methanol-to-propylene (MTP) processes. In MTO, methanol is converted primarily to ethylene and propylene in the presence of a molecular sieve catalyst. In MTP, methanol is dehydrated to produce dimethyl ether, which is then converted to propylene. Both processes involve complex operations downstream of the reactor(s) to purify the product, capture unconverted reagents for recycle, and purge contaminants. Typically, low temperature partial condensation is involved, and at least a portion of the uncondensed gas is recycled in the process.

In non-polymeric olefin derivative manufacturing, an olefin and other reagents are introduced into a high-pressure reactor. The raw effluent from the reactor is transferred continuously to one or more separation steps, from which a stream of raw derivative product is withdrawn for further purification. A stream of overhead gases, containing unreacted olefin, is also withdrawn from the separation steps and is recirculated back to the reactor.

Both of these types of manufacturing operations need to vent a portion of uncondensed gas to prevent build-up of unwanted contaminants in the reaction loop. However, the vented overhead gas typically contains unreacted olefin that, without further treatment, would otherwise go to waste.

Additionally, polyethylene (PE) and polypropylene (PP) are two of the most demanded polymers in the world. Together, these polymers make up half of the volume of plastic produced worldwide.

During polyolefin production, a small portion of the olefin feedstock is lost through raw material purification, chemical reaction, and product purification and finishing. In particular, paraffin that enters with the olefin feedstock must be removed to prevent its build up in the reactor loop, and olefin is lost when this paraffin is purged from the loop. This results in an annual loss of $1 million to $3 million per year for a typical polyolefin plant. The development of a more efficient way to prevent the loss of olefin monomer in the feedstock has been an on-going process for those in the petrochemical field.

In polyolefin manufacturing, a feedstock containing olefin monomer, catalysts, and other agents is introduced into a high-pressure polymerization reactor. During the reaction, a raw polymer product is produced. The raw product contains polyolefin, significant amounts of unreacted olefin, and small amounts of solvents, catalysts, stabilizers, other hydrocarbons or any other materials, depending on the manufacturing process used. To remove the volatile contaminants dissolved in the raw product, it is passed to large bins, where nitrogen is used to purge them out. The vent gas from this step contains nitrogen, unreacted olefin monomer, unwanted analogue paraffins that entered with the olefin feedstock, and other process-specific materials. In the past, this vent gas was sent for flaring, resulting in a waste of unreacted olefin.

Various process and techniques have been proposed for mitigating the loss of unreacted olefin in a variety of streams.

U.S. Pat. No. 4,623,704, to Dembicki et al. (Dow Chemical Company), discloses a process for treating a polymerization vent gas with a membrane. The vent stream is compressed and then cooled and condensed. Cooled gas and liquid are sent to a liquid/gas separator. After separation, the gas stream is sent through a series of membrane separation steps, which produce a permeate stream enriched in ethylene. The recovered ethylene is recycled to the polymerization process.

Co-owned U.S. Pat. Nos. 5,089,033 and 5,199,962, to Wijmans (Membrane Technology and Research, Inc.), disclose processes for recovering a condensable component in a gas stream that would otherwise be discharged into the atmosphere. The processes involve a condensation step and a membrane separation step. In one embodiment, the gas stream is compressed and cooled to carry out the condensation step. Uncondensed gas is then passed across a membrane that is selectively permeable to the condensable component.

Co-owned U.S. Pat. No. 6,271,319, to Baker et al. (Membrane Technology and Research, Inc.), discloses a process for treating the uncondensed gas stream using a gas separation membrane that is permeable for propylene over propane. A permeate stream enriched in olefin is withdrawn and recycled to the reactor inlet.

These patents, and other prior art technologies, have mainly focused on condensing a gas stream and recovering unreacted olefin from the resulting uncondensed gas produced from the condensation step. However, little is taught on recovering unreacted olefins from the condensed liquid stream.

Co-owned U.S. Pat. No. 5,769,927, to Gottschlich et al. (Membrane Technology and Research, Inc.), discloses a process for treating a purge vent stream from a polymer manufacturing operation. The purge vent stream contains an unreacted olefin monomer and nitrogen. The purge vent stream is initially treated in a condensation step. The uncondensed gas is then passed to a membrane separation step, where the membrane is organic-selective, meaning that the membrane is selective for unreacted monomer over other gases. The liquid condensate is directed to a flash evaporation step. The flashing step produces a liquid product stream enriched in monomer and a flash gas that is recirculated in the process.

Despite the above improvements, there remains a need for better olefin recovery technology applicable to processes that make or use olefins or polyolefins.

### SUMMARY OF THE INVENTION

Accordingly, the present invention relates to a process for recovering olefins by treating an effluent gas stream comprising an olefin, a paraffin, and a third gas. The effluent stream is withdrawn from either an olefin or an olefin derivative manufacturing operation. During treatment, the effluent gas stream is condensed and separated, producing a liquid condensate stream and an uncondensed gas stream. Both of these streams contain olefin along with other components, such as paraffin and a third gas. To recover the unreacted olefin, the liquid condensate stream is treated by a membrane separation step. Recovered unreacted olefin from treating the condensate may be sent in a recycle loop for use as feedstock back in the manufacturing operation.

Therefore, in a basic embodiment, the process of the invention includes the following steps:
(a) passing said effluent gas stream to a compressor to produce a compressed gas stream;
(b) partially condensing the compressed stream, including cooling and separating the compressed stream into an uncondensed gas stream depleted in olefin and paraffin and a condensed liquid condensate enriched in olefin and paraffin; and
(c) separating the condensed liquid condensate using a membrane to produce an olefin-enriched permeate stream and an olefin-depleted residue stream.

In certain embodiments, the effluent gas stream of step (a) may arise from the following three types of manufacturing operations:

The first type is an operation that produces olefins. These operations include, but are not limited to, fluid catalytic cracking, olefin cracking, steam cracking, olefin metathesis, a methanol-to-olefin process (MTO), and a methanol-to-propylene (MTP) process.

The second type is an operation that manufactures a non-polymeric olefin derivative, using olefins as a feedstock. Non-limiting examples of these operations include chlorohydrin production, butyraldehyde production, oxo alcohol production, isopropyl alcohol production, acrylic acid production, allyl chloride production, allyl alcohol production, acrylonitrile production, cumene production, ethylene oxide production, vinyl acetate production, ethylene dichloride production, ethanol production, and ethylbenzene production.

The third type is an operation that produces a polyolefin. In a polyolefin manufacturing plant, the effluent stream is vented from a purge bin. The effluent stream from this type of process is referred to herein as a “purge stream.”

In all cases, the effluent gas stream comprises an olefin, an analogous paraffin, and a third gas. In certain embodiments, the olefin is ethylene or propylene. In other embodiments, the olefin is butylene. The effluent gas stream may also comprise multiple sets of olefins and analogous paraffins, for example, ethane/ethylene and propane/propylene. The effluent gas stream also contains a third gas, such as methane, hydrogen, or nitrogen.

The goal of steps (a) and (b) is to bring the effluent gas stream to a pressure/temperature condition beyond the dewpoint of the olefin to be recovered, so that a portion of the olefin will condense out of the gas stream. Thus, the separation of the compressed stream creates a liquid condensate and an uncondensed (residual) gas stream. The condensate is enriched in olefin and paraffin and the uncondensed gas stream depleted in olefin and paraffin relative to the effluent stream.

The condensation step usually involves chilling and compression. Compressing the gas raises the dewpoint temperature, so a combination of compression and chilling is generally preferred.

In certain embodiments, the conditions of the process may be such that the effluent gas stream is already at high pressure. In this case, chilling alone may suffice to induce condensation, and the compression step may be dispensed with.

In step (c), the liquid condensate from condensation step (b) is treated in a membrane separation step, which may be carried out under pervaporation or vapor permeation conditions. The membrane in this step is selective for olefin over paraffin. The membrane separation of step (c) thus results in a permeate stream enriched in olefin and a residue stream depleted in olefin.

Membranes for use in step (c) of the process of the invention may comprise any material suitable for preferentially permeating olefin over paraffin. Preferably, the membrane is an inorganic membrane. In certain embodiments, the membrane preferably exhibits an olefin permeance of at least 400 gpu.

Step (c) may take the form of a single membrane separation operation or of multiple sub-operations, depending on the feed composition, membrane properties, and desired results.

In certain embodiments, the olefin-depleted residue stream is further separated using a second membrane separation step to produce a second olefin-enriched permeate stream and a second olefin-depleted residue stream. The second olefin-enriched permeate stream may then be recycled within the process either upstream of step (a) or to a point a point after step (a), but upstream of step (c). In the latter case, a second compressor would be needed to recompress the second olefin-enriched permeate stream.

Also disclosed herein is an apparatus for either treating an effluent gas stream arising from an operation that manufactures olefins or non-polymeric olefin derivatives or a purge gas stream arising from a polymer manufacturing operation. The apparatus is designed to perform the processes of the invention. In a basic embodiment, the apparatus comprises the following components:
(a) a compressor having a feed gas inlet and a compressed gas outlet;
(b) a condenser having a compressed gas inlet and a cooled gas outlet, wherein the compressed gas outlet of the compressor is in gas communication with the compressed gas inlet;
(c) a phase separator having a cooled gas inlet, an uncondensed gas outlet, and a condensed gas outlet, wherein the cooled gas outlet of the condenser is in fluid communication with the cooled gas inlet; and
(d) a membrane separation unit having a feed inlet, a residue outlet, and a permeate outlet, wherein the condensed gas outlet is in fluid communication with the feed inlet.

In some embodiments, the apparatus may also include a vaporizer unit when the membrane separation unit operates under vapor permeation conditions. In other embodiments, where the effluent gas stream is already at high pressure, the compressor may be located downstream of the membrane separation unit so that the feed gas inlet can accept gas from the permeate outlet.

It is to be understood that the above summary and the following detailed description are intended to explain and illustrate the invention without restricting it in scope.

### BRIEF DESCRIPTION OF THE DRAWINGS

FIG. 1 is a schematic drawing showing an olefin recovery process comprising a membrane separation step according to a basic embodiment of the invention.

FIG. 2 is a schematic drawing showing an olefin recovery process comprising two membrane separation steps according to a basic embodiment of the invention.

FIG. 3 is a schematic drawing showing an olefin recovery processing where the source of the feed gas is already at high pressure according to a basic embodiment of the invention.

FIG. 4 is a schematic drawing of a basic embodiment of an olefin recovery apparatus that includes a compressor, a condenser, a phase separator, and two membrane separation units.

FIG. 5 is a schematic drawing showing an olefin recovery process without the use of a membrane separation step not in accordance with the invention.

### DETAILED DESCRIPTION OF THE INVENTION

The term “effluent gas stream” as used herein is construed as including a gas stream withdrawn from any unit operation or operations during an olefin or an olefin-derivative manufacturing operation.

The term “olefin” as used herein means a member of the family of unsaturated hydrocarbons having a carbon-carbon double bond of the series CₙH₂ₙ, including members in which at least one halogen atom has been substituted for one of the hydrogen atoms.

The term “non-polymeric olefin derivative” as used herein refers to a product made from at least one olefin, wherein the product does not contain repeating units of the olefin derivative monomer. Examples of propylene derivatives include, but are not limited to, chlorohydrin (a precursor of propylene oxide); butyraldehyde (a precursor to butyl alcohol); oxo alcohols, such as 2-methyl-2-butanol, n-butanol, 2-ethylhexanol, isononyl alcohol, and isodecyl alcohol; isopropyl alcohol; acrylic acid; allyl chloride; acrylonitrile; and cumene. Examples of ethylene derivatives include, but are not limited to, ethylene oxide, vinyl acetate, ethylene dichloride, ethanol, and ethylbenzene.

The term “C₂₊ hydrocarbon” means a hydrocarbon having at least two carbon atoms.

The invention relates to an improved process for recovering unreacted olefin from an effluent gas stream, comprising an olefin, a paraffin, and a third gas that arises from an olefin or olefin-derivative manufacturing operation. The process also provides for selectively purging paraffin from the reactor loop. By a reactor loop, we mean a configuration in which at least a part of the effluent or purge gas stream from the reactor is recirculated directly or indirectly to the reactor. The process can be applied to any loop in which olefin is fed to the reactor, and in which olefin and paraffin are present in the effluent or purge gas steam from the reaction loop.

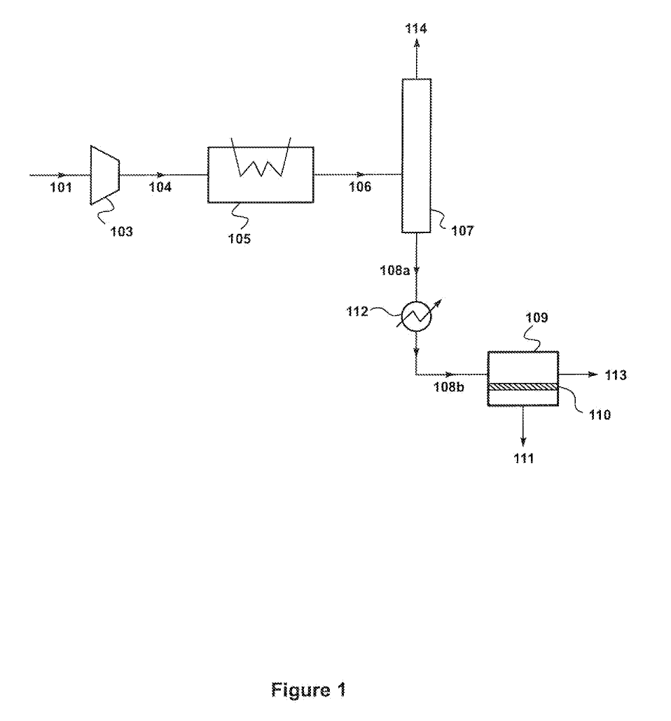

It will be appreciated by those of skill in the art that FIG. 1 and the other figures showing process schemes herein are very simple block diagrams, intended to make clear the key unit operations of the embodiment processes of the invention, and that actual process trains may include many additional steps of standard type, such as heating, chilling, compressing, condensing, pumping, various types of separation and/or fractionation, as well as monitoring of pressures, temperatures, flows, and the like. It will also be appreciated by those of skill in the art that the details of the unit operations may differ from process to process.

A basic embodiment of the olefin recovery process is shown in FIG. 1.

A feed gas stream, 101, from a manufacturing process typically contains at least an unreacted olefin, an analogous paraffin, and a third gas. For purposes of FIG. 1 and the following Examples, the feed gas stream is assumed to be a purge gas stream arising from a polymer manufacturing operation. However, as discussed above, the feed gas stream may also be an effluent gas stream that is withdrawn from either a manufacturing operation that produces olefins or a manufacturing operation that uses olefins as a feedstock to produce non-polymeric olefin-derivatives.

Such non-limiting examples of processes that produce olefins include fluid catalytic cracking, olefin cracking, steam cracking, olefin metathesis, a methanol-to-olefin process (MTO), and a methanol-to-propylene (MTP) process. A reference that provides discussion of design and operation of modern FCC units, a typical source of low-molecular weight olefins, is described in Chapter 3 of “Handbook of Petroleum Refining Processes” Second Edition, R. A. Meyers (Ed), McGraw Hill, 1997, incorporated by reference herein. The other processes are well known in the art and do not require any lengthy description herein.

For olefin-derivative manufacturing processes, non-limiting examples include the production of chlorohydrin (a precursor of propylene oxide), butyraldehyde (a precursor of butyl alcohol), isopropyl alcohol, acrylic acid, allyl chloride, acrylonitrile, cumene, ethylene oxide, vinyl acetate, ethylene dichloride, ethanol, and ethylbenzene.

The third gas in the purge gas stream is typically, but not always, methane or an inorganic gas, such as hydrogen, nitrogen or argon. The third gas also has a lower boiling point than both the principal olefin and the principal paraffin in the purge gas stream. Gases of this type are inevitably present in streams coming from the operations in the manufacturing train, often because they are carried in as unwanted contaminants with the feedstock, and sometime because they are used in the reactors or the product purification steps and have intrinsic value in the manufacturing process if they could be separated and recovered.

The ratio of olefin to paraffin in the stream may be as much as 5:1, 6:1 or even 7:1 or more. If this stream were to be vented from the manufacturing process without further treatment, then as many as five, six, or seven volumes of olefin would be lost for every volume of paraffin that is purged.

Returning to FIG. 1, a purge gas stream, 101, is routed to compression step 103, the goal of which is to compress the stream to a pressure which the gas mixture may be partially condensed in the subsequent process steps. The compression step may be carried out using compression equipment of any convenient type, and may be performed in one stage or in a multistage compression train, depending on the degree of compression needed. It is preferred that the pressure to which stream 101 is raised be no more than about 35 bara, and more preferably no more than about 30 bara.

The stream emerging from compression step 103 is compressed stream 104. This stream is sent to a condensation step, 105. The condensation step includes cooling of stream 104 to below the olefin dewpoint temperature, such that a major portion of the olefin is condensed, followed by separation of the resulting liquid and gas phases. Cooling may be performed in any manner, and in one or more sub-steps, including, but not limited to, simple air or water aftercooling of the compressor outlet gases, heat exchange against other on-site process streams, chilling by external refrigerants, and any combinations of these. Preferably, this step should cool stream 104 to a temperature no lower than −40° C., and yet more preferably to no colder than about −35° C.

The liquid and gas phases that are formed by compression and cooling are separated by conventional means in a simple phase separator, knock-out drum or the like, 107, to yield condensed liquid stream, 108a, and uncondensed gas stream, 114. The condensed liquid stream 108a typically comprises 80 mol %, 90 mol %, or more olefin and paraffin. Uncondensed gas stream, 114, is depleted in olefin and paraffin and may be sent for further treatment to recover any unreacted olefin, reused in the purge bin, or passed to any convenient destination.

For membrane separation step 109, any membrane with suitable performance properties may be used. The membrane, 110, may take the form of a homogeneous film, an integral asymmetric membrane, a multilayer composite membrane, a membrane incorporating a gel or a liquid layer or particulates, or any other form known in the art.

The membrane or membranes to be used in step 109 are made of any material suitable for selectively permeating olefin over paraffin. Preferably, the membranes provide propylene/propane selectivity of at least 5 and propylene flux of 400 gpu under favorable conditions. For ethylene/ethane separation, the preferred selectivity of the membrane is 3 and the preferred ethylene flux is 400 gpu. For butylene/butane separation, the preferred selectivity of the membrane is at least 5 and the preferred ethylene flux is 400 gpu.

These membranes are preferably inorganic membranes. Inorganic membranes with olefin/paraffin separating properties are very finely porous and act as very fine sieves that separate on the basis of polarity difference. Inorganic membranes are characterized by good temperature and chemical resistance. More preferably, the inorganic membranes are zeolite membranes. Such membranes include, but are not limited to, zeolite-based membranes that are crystalline oxides consisting of silicon, aluminum, and other cations, such as sodium and potassium coated on ceramic or other types of support structures.

In some embodiments, membranes for separating olefin and paraffins include polymeric membranes. Typically, these membranes have a selective layer made from a glassy polymer. Representative examples of these membranes include, but are not limited to, poly(phenylene oxide) (PPO), polyimides, perflourinated polyimides, Hyflon® AD, and Cytop®.

In other embodiments, the membranes used in step 109 may include facilitated transport membranes. These contain a liquid that itself contains, for example, free silver ions that react selectively and reversibly with unsaturated hydrocarbons, to selectively carry olefin (propylene) across the membrane.

The membranes may be manufactured as flat sheets or as hollow fibers and housed in any convenient module form, including spiral-wound modules, tubular modules, plate-and-frame modules, and potted hollow-fiber modules. The making of all these types of membranes and modules is well-known in the art.

The membrane separation steps disclosed herein may be carried out using a single membrane module or a bank of membrane modules or an array of modules. A single unit or stage containing on or a bank of membrane modules is adequate for many applications. If either the residue or permeate stream, or both, requires further olefin removal, it may be passed to a second bank of membrane modules for a second processing step. Such multi-stage or multi-step processes, and variants thereof, will be familiar to those of skill in the art, who will appreciate that the membrane separation step may be configured in many possible ways, including single-stage, multistage, multistep, or more complicated arrays of two or more units, in serial or cascade arrangements.

The membrane separation steps disclosed herein can be operated by any mechanism that provides a driving force for transmembrane permeation. Most commonly, this driving force is provided by maintaining a pressure difference between the feed and permeate sides, or by sweeping the permeate side continuously with a gas that dilutes the permeating species, both of which techniques are well known in the membrane separation arts.

In FIG. 1, the membrane separation step, 109, occurs under vapor permeation conditions. Liquid stream 108a is heated by heater 112 of any convenient type to produce a heated vaporized stream, 108b, before flowing across the feed side of membrane 110.

In other embodiments, membrane separation step 109 may occur under pervaporation conditions. By “pervaporation conditions” we mean that the feed is heated to elevate its vapor pressure but maintained at a sufficiently high pressure to prevent evaporation on the feed side of the membrane. The permeate side is maintained at a pressure substantially below the vapor pressure of the feed so vapor will permeate the membrane. If membrane separation step 109 occurs under pervaporation conditions, liquid stream 108a is first heated and then flows to and across the feed side of membrane 110. The low pressure permeate vapor, enriched in the more permeable component, may optionally be cooled and condensed or may be compressed and condensed or a combination of the two.

Under either condition, a residue stream, 113, that is depleted in olefin relative to stream 108b, is withdrawn from the feed side of the membrane. The membrane separation step reduces the olefin content of this stream, preferably to the point that the ratio of olefin to paraffin in the stream is reduced to about 1:1, and more preferably below 1:1. This stream may be purged from the process with comparatively little loss of olefin.

The permeate stream, 111, is enriched in olefin compared with the membrane feed. Optionally, in certain embodiments, this stream may be used as a coolant for heat recovery at various locations within the process to minimize refrigerant energy usage. For example, permeate stream 111 may be used as a coolant in the heat-exchange/condensation step 105, emerging as warmed permeate stream.

Alternatively, if membrane separation step 109 takes place under pervaporation conditions, it may be more beneficial to cool and condense stream 111 to provide or augment the driving force for the pervaporation step.

Permeate stream 111 represents a substantial source of recovered olefin, preferably containing a chemical grade olefin, having an olefin content of at least 90%. In a preferred embodiment, permeate stream 111 is returned as feedstock to the manufacturing reactor. In this case, the permeate stream most preferably contains a polymer grade olefin, having an olefin content of about 99% or above, such as 99.5%.

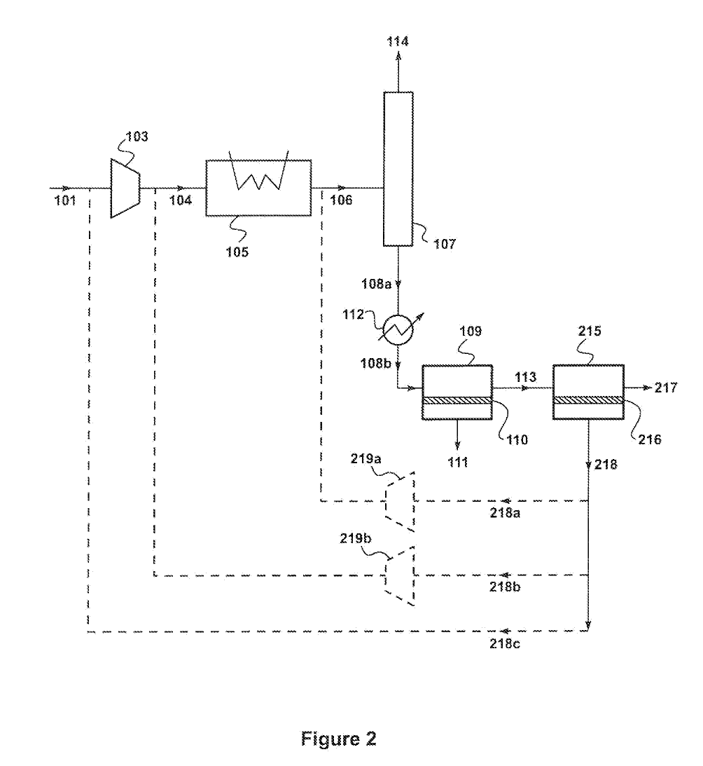

Another embodiment of the olefin recovery process is shown in FIG. 2. This embodiment is similar to that of FIG. 1 in that the condensed liquid stream 108a undergoes membrane separation.

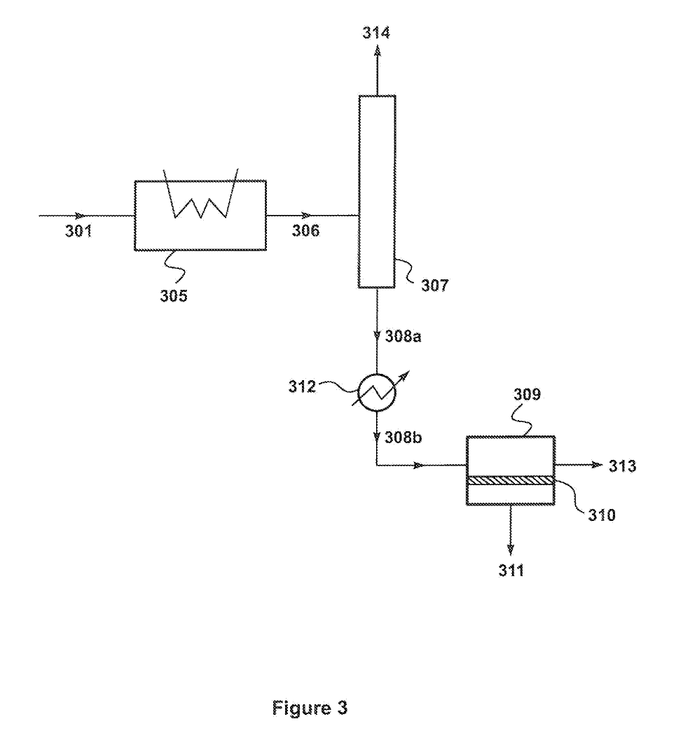

In this case, however, residue stream, 113, which is depleted in olefin relative to stream 108b, is further treated by a second membrane separation step, 215. Stream 113 is passed as a feed stream across membrane 216 that is selectively permeable to olefin over paraffin. The residue stream, 217, contains a major part/most of the paraffin in the feed gas stream 101 and is purged from the process. The permeate stream, 218, is enriched in olefin and may be recycled to a number of locations within the process. Non-limiting examples of these locations are shown in FIG. 3 and indicated by permeate streams 218a, 218b, and 218c. Stream 218a may be recycled upstream of the separator, 107, but downstream of the condenser, 105, to mix with cooled stream 106. Stream 218b may be recycled upstream of the condenser, 105, but downstream of the compressor, 103, to be mixed with compressed stream 104. In these two situations, permeate stream 218a or 218b would have to be recompressed by additional compressors 219a or 219b, respectively. Lastly, stream 218c may be recycled upstream of compressor 103 to be mixed with feed stream 101.

Preferred membranes for second membrane separation step 215 are inorganic membranes, similar to those used in membrane separation step 109, described above.

Another embodiment of the olefin recovery process is shown in FIG. 3. Here, an effluent gas stream, 301, is at a high enough pressure coming from a non-polymeric olefin derivative manufacturing operation that no compression is needed. Thus, stream, 301, can be sent directly to a condensation step, 305. The condensation step includes cooling of stream 301 to below the olefin dewpoint temperature, such that a major portion of the olefin is condensed, to produce a cooled stream, 306.

Cooled stream 306 is then separated into liquid and gas phases. The liquid and gas phases that are formed by compression and cooling are separated by conventional means in a knock-out drum or the like, 307, to yield condensed liquid stream, 308a, and uncondensed gas stream, 314.

Condensed liquid stream 308a is heated by heater, 309, of any convenient type to produce a vapor stream 308b. In the alternative, stream 308a could be vaporized using a lower temperature heat source by reducing the pressure on the stream by means of a valve or the like.

Vapor stream 308b is then passed as a feed stream to a membrane separation step, 309. Membrane or membranes, 310, to be used in step 309 are inorganic membranes, but any other material suitable for selectively permeating olefin over paraffin may be used. A residue stream, 313, that is depleted in olefin relative to stream 308b, is withdrawn from the feed side of the membrane. This stream may be purged from the process with comparatively little loss of olefin. A permeate stream, 311, enriched in olefin compared to stream 308b, is withdrawn from the permeate side of the membrane and may be recycled back to the manufacturing reactor or sent for further processing, optionally after recompression.

Membrane separation step 309 reduces the olefin content of stream 313, preferably to the point that the ratio of olefin to paraffin in the stream is reduced to about 1:1, and more preferably below 1:1.

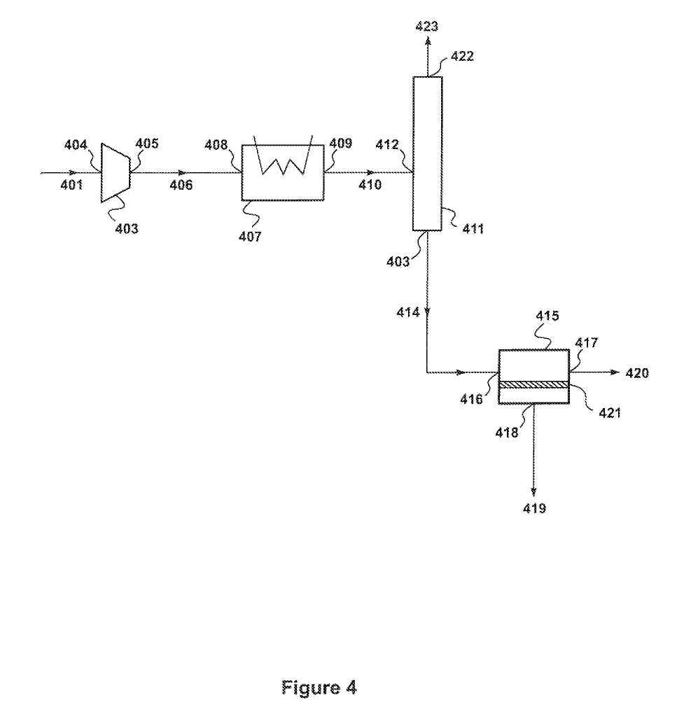

FIG. 4 is a schematic drawing of an apparatus for recovering olefin in a manufacturing operation. The apparatus comprises a compressor, 403, a condenser, 407, a phase separator, 411, and a membrane separation unit, 415.

In operation, an effluent or purge gas stream, 401, comprising an olefin, a paraffin, and a third gas, is introduced into a compressor, 403, via feed gas stream inlet, 404. The compressor produces a compressed gas stream, 406, that exits the compressor through compressed gas outlet, 405.

The condenser, 407, may include any type of industrial chiller, heat exchanger or refrigeration unit capable of lowering the temperature of gas stream 406 to the point that at least partial condensation of olefins and paraffins occurs. The condenser further comprises a compressed gas inlet, 408, and a cooled gas outlet, 409. The compressed gas, 406, is directed into the compressed gas inlet, 408, of the condenser where it is cooled to below the olefin dewpoint temperature, such that a major portion of the olefin is condensed. Once cooled, a cooled stream, 410, exits the condenser through cooled gas outlet, 409.

The phase separator, 411, comprises a cooled stream inlet, 412, a condensed stream outlet, 413, and an uncondensed stream outlet, 422. The phase separator, 411, separates the liquid and gas portions of cooled stream 410. Phase separator 411 may be of any type known in the art, including, but not limited to a horizontal separator, a vertical separator, or a cyclone separator. The cooled stream inlet, 412, is in fluid communication with cooled stream outlet, 409. After separation, a condensed stream, 414, and an uncondensed gas stream, 422, exit the phase separator through outlets 413 and 422, respectively.

The membrane separation unit, 415, includes a feed inlet, 416, a permeate outlet, 418, and a residue outlet, 417. Membrane separation unit 415 is in fluid communication with condensed gas stream outlet 413 of phase separator 411. The feed inlet, 416, allows condensed stream 414 to enter the membrane separation unit, 415. Condensed stream 414 may enter the membrane separation unit, 415, as a liquid or gas depending on the separation conditions.

In embodiments where the separation in membrane separation unit 415 occurs under vapor permeation, a vaporizer unit, having a liquid inlet in fluid communication with condensed gas stream outlet 413 and a gas outlet in gas communication with the feed inlet, 416, is used to vaporize the condensed stream.

The condensed stream, 414, is treated by membrane separation unit, 415, which contains membrane 421 that is selectively permeable to olefin over paraffin. Separation unit 415 produces a permeate stream, 419, and a residue stream, 420. The residue outlet, 417, and permeate outlet, 418, allow for the residue and permeate streams to be withdrawn from the separation unit.

The invention is now further described by the following examples, which are intended to be illustrative of the invention, but are not intended to limit the scope or underlying principles in any way.

### Examples

### Example 1

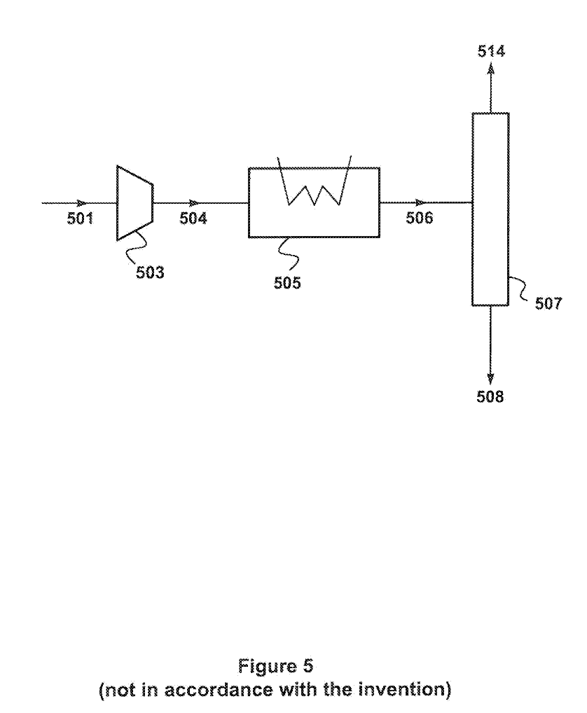

### Treatment of a Purge Gas Stream Using the Process of FIG. 5 (not in Accordance with the Invention)

For comparison with the following examples, a calculation was performed in a process where the liquid condensate from a separator was not treated by membrane separation.

For the calculation, the purge gas stream was assumed to have a flow rate of 1,141 kg/hour and contain propylene, propane, and nitrogen. It was also assumed that the molar compositions were approximately as follows:

### Nitrogen: 78%

### Propylene: 19%

### Propane: 3%

It was further assumed that the purge gas stream was compressed to 24 bara in compression step 503, then cooled to −20° C. in condensation step 505.

The calculation was performed using differential element membrane code written at MTR and incorporated into a computer process simulation program (ChemCad 6.3, ChemStations, Austin, Tex.).

Referring to FIG. 5, a purge stream, 501, is routed to a compression step, 503. The stream emerging from compression step 503 is a compressed stream, 504.

Compressed stream 503 is directed to a condensation step 505. The condensation step includes cooling of stream 504 to below the olefin dewpoint temperature, such that a major portion of the olefin is condensed, followed by separation of the resulting liquid and gas phases in cooled stream 506. The liquid and gas phases that are formed by compression and cooling are separated by conventional means in a knock-out drum or the like. 507, to yield a condensed liquid stream, 508, and an uncondensed gas stream, 514.

The results of the calculations are shown in Table 1:

TABLE 1

| | Stream | | | | |
|---|---|---|---|---|---|
| | 501 | 504 | 506 | 514 | 508 |
| Total Mass flow | 1,101 | 1,101 | 1,101 | 1,005 | 96 |
| (kg/h) | | | | | |
| Temp | 70 | 90 | −20 | −20 | −20 |
| (° C.) | | | | | |
| Pressure | 1 | 24 | 24 | 24 | 24 |
| (bara) | | | | | |

Component (mol %)

| | | | | | |
|---|---|---|---|---|---|
| Nitrogen | 77.9 | 77.9 | 77.9 | 83.0 | 3.7 |
| Propylene | 19.1 | 19.1 | 19.1 | 14.8 | 82.0 |
| Propane | 3.0 | 3.0 | 3.0 | 2.2 | 14.3 |

Mass flow (kg/h)

| | | | | | |
|---|---|---|---|---|---|
| Nitrogen | 770 | 770 | 770 | 768 | 2 |
| Propylene | 284 | 284 | 284 | 205 | 79 |
| Propane | 47 | 47 | 47 | 33 | 14 |

With neither of the uncondensed gas stream, 514, nor the condensate stream, 508, being treated, the process produces a condensate stream where the ratio of olefin to paraffin is about 6:1. No olefin is recovered.

### Example 2

### Olefin Recovery Process in Accordance with the Invention of FIG. 1

A calculation was performed to model the performance of the process of FIG. 1 in treating a purge gas stream in a polymer manufacturing operation. The membrane separation of the condensate occurred under vapor permeation conditions.

The results of the calculations are shown in Table 2.

TABLE 2

| Stream | 101 | 104 | 106 | 114 | 108a | 108b | 111 | 113 |
|---|---|---|---|---|---|---|---|---|
| Total Mass | 1,101 | 1,101 | 1,101 | 1,005 | 96 | 96 | 73 | 23 |
| flow (kg/h) | | | | | | | | |
| Temp (° C.) | 70 | 90 | −20 | −20 | −20 | 80 | 77 | 75 |
| Pressure | 1 | 24 | 23 | 23 | 23 | 5 | 1.1 | 5 |
| (bara) | | | | | | | | |

Component (mol %)

| | | | | | | | | |
|---|---|---|---|---|---|---|---|---|
| Nitrogen | 77.9 | 77.9 | 77.9 | 83.0 | 3.7 | 3.7 | 0.2 | 14.5 |
| Propylene | 19.1 | 19.1 | 19.1 | 14.8 | 82.0 | 82.0 | 95.0 | 42.7 |
| Propane | 3.0 | 3.0 | 3.0 | 2.2 | 14.3 | 14.3 | 4.8 | 42.8 |

Mass flow (in kg/h)

| | | | | | | | | |
|---|---|---|---|---|---|---|---|---|
| Nitrogen | 770 | 770 | 770 | 768 | 2 | 2 | 0 | 2 |
| Propylene | 284 | 284 | 284 | 205 | 79 | 79 | 69 | 10 |
| Propane | 47 | 47 | 47 | 33 | 14 | 14 | 4 | 11 |

Using just one membrane to treat the vaporized condensate, 108b, the process achieves only 24% recovery of olefin, but the olefin to paraffin ratio in the purge/residue stream, 113, is reduced to about 1:1. In addition, the propylene purity in stream 111 is about 95%.

### Example 3

### Olefin Recovery Process in Accordance with the Invention of FIG. 2

A calculation was performed to model the performance of the process of FIG. 2 in treating a purge gas stream in a polymer manufacturing operation. The membrane separation of the condensate occurred under vapor permeation conditions.

The results of the calculations are shown in Table 3.

TABLE 3

| Stream | 101 | 104 | 106 | 114 | 108a | 108b | 111 | 113 | 218c | 217 |
|---|---|---|---|---|---|---|---|---|---|---|
| Total Mass | 1,101 | 2,864 | 2,864 | 955 | 1,909 | 1,909 | 52 | 1,857 | 1,763 | 93 |
| flow (kg/h) | | | | | | | | | | |
| Temp (° C.) | 70 | 90 | −20 | −20 | −20 | 80 | 80 | 80 | 76 | 72 |
| Pressure | 1 | 24 | 23 | 23 | 23 | 5 | 1 | 5 | 1 | 5 |
| (bara) | | | | | | | | | | |

Component (mol %)

| | | | | | | | | | | |
|---|---|---|---|---|---|---|---|---|---|---|
| Nitrogen | 77.9 | 35.8 | 35.8 | 82.8 | 3.7 | 3.7 | 0.1 | 3.8 | 0.4 | 56.5 |
| Propylene | 19.1 | 61.2 | 61.2 | 16.5 | 91.8 | 91.8 | 99.3 | 91.6 | 96.7 | 12.4 |
| Propane | 3.0 | 3.0 | 3.0 | 0.7 | 4.5 | 4.5 | 0.6 | 4.6 | 2.9 | 31.1 |

Mass flow (in kg/h)

| | | | | | | | | | | |
|---|---|---|---|---|---|---|---|---|---|---|
| Nitrogen | 770 | 775 | 775 | 727 | 47 | 47 | 0 | 47 | 5 | 42 |
| Propylene | 284 | 1,989 | 1,989 | 218 | 1,771 | 1,771 | 52 | 1,719 | 1,705 | 14 |
| Propane | 47 | 101 | 101 | 10 | 91 | 91 | 0 | 91 | 54 | 37 |

Using two membrane separation steps to treat the vaporized condensate, 108b, the process achieves only 18% recovery of olefin, but the olefin to paraffin ratio in the purge/second residue stream, 217, is reduced to about 1:3. In addition, the propylene purity in stream 111 is about 99%.

We claim:

1. A process for treating an effluent gas stream arising from an operation that manufactures an olefin or an olefin derivative, said effluent gas stream comprising an olefin, a paraffin and a third gas, the process comprising the steps of:
(a) passing said effluent gas stream to a compressor to produce a compressed stream;
(b) partially condensing the compressed stream, including cooling and separating the compressed stream into an uncondensed gas stream depleted in olefin and paraffin and a condensed liquid condensate enriched in olefin and paraffin; and
(c) separating the condensed liquid condensate using a first membrane to produce a first olefin-enriched permeate stream and a first olefin-depleted residue stream.

2. The process of claim 1, wherein the olefin is selected from the group consisting of ethylene, propylene and butylene.

3. The process of claim 1, wherein the operation is selected from the group consisting of steam cracking, fluid catalytic cracking, propane dehydrogenation, olefin metathesis, a methanol-to-olefin process, a methanol-to-propylene process, and polyolefin manufacturing.

4. The process of claim 1, wherein the first membrane is an inorganic membrane.

5. The process of claim 1, wherein the condensed liquid condensate is revaporized prior to step (c).

6. The process of claim 1, wherein the third gas is nitrogen or hydrogen.

7. The process of claim 1, further comprising the step of:
(d) separating the first olefin-depleted residue stream using a second membrane to produce a second olefin-enriched permeate stream and a second olefin-depleted residue stream; and
(e) returning the second olefin-enriched permeate stream upstream of step (a).

8. The process of claim 1, further comprising the steps of:
(d) separating the first olefin-depleted residue stream using a second membrane to produce a second olefin-enriched permeate stream and a second olefin-depleted residue stream;
(e) compressing the second olefin-enriched permeate stream to produce a compressed permeate stream; and
(f) returning the compressed permeate stream downstream of step (a), but upstream of step (c).

9. The process of claims 7 or 8, wherein the second membrane is an inorganic membrane.

10. A process for treating an effluent gas stream arising from an operation that manufactures an olefin or an olefin derivative, said effluent gas stream comprising an olefin, a paraffin and a third gas, the process comprising the steps of:
(a) partially condensing the effluent gas stream, including cooling and separating the effluent gas stream into a condensed liquid condensate enriched in olefin and paraffin and an uncondensed gas stream depleted in olefin and paraffin; and
(b) separating the condensed liquid condensate from step (a) using a first membrane to produce a first olefin-enriched permeate stream and a first olefin-depleted residue stream;

11. The process of claim 10, wherein the first membrane is an inorganic membrane.

12. The process of claim 10, wherein the condensed liquid condensate is revaporized prior to step (b).

13. The process of claim 10, further comprising the steps of:
(c) separating the first olefin-depleted residue stream using a second membrane to produce a second olefin-enriched permeate stream and a second olefin-depleted residue stream;
(d) compressing the second olefin-enriched permeate stream to produce a compressed permeate stream; and
(e) returning the compressed permeate stream downstream of step (a), but upstream of step (c).

14. The process of claim 13, wherein the second membrane is an inorganic membrane.
